(19) United States
(12) Patent Application Publication
(10) Pub. No.: US 2004/0241477 A1
(43) Pub. Date: Dec. 2, 2004

(54) Laminate film, sealant film and package

(76) Inventor: Kazuyuki Watanabe, Oita-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/491,018

(22) PCT Filed: Sep. 25, 2002
(86) PCT No.: PCT/JP02/09833

Related U.S. Application Data
(60) Provisional application No. 60/342,099, filed on Dec. 26, 2001.

(30) Foreign Application Priority Data
Sep. 28, 2001 (JP) 2001-303212

(51) Int. Cl. A61B 5/00

(52) U.S. Cl. 428/515; 600/365; 523/118

(57) ABSTRACT

A resin composition layer [layer (A)] contains (a) from 30 to 60 mass % of a linear low-density polyethylene produced using a metallocene catalyst or a single site-type catalyst, (b) from 10 to 30 mass % of a low-density polyethylene and (c) from 10 to 40 mass % of at least one resin selected from the group consisting of an ethylene-propylene copolymer, an ethylene-butene copolymer, a propylene-butene copolymer, an ethylene-propylene-butene terpolymer and polybutene-1 [provided that (a)+(b)+(c)=100 mass %]. A high-density polyethylene layer [layer (B)] contains (d) a high-density polyethylene having a density of 0.950 to 0.970 g/cm³. At least two layers of layer (A) and layer (B) are comprised.

### CROSS-REFERENCE TO RELATED APPLICATIONS

This claims the benefit of provisional patent application No. 60/342,099, and Japanese Patent Application No. 2001-303212.

### TECHNICAL FIELD

The present invention relates to a laminate film suitable as a food or medical packaging material which is excellent in easy openability, whitening property, water vapor barrier property and low-temperature heat sealability and has a wide heat seal temperature width, and also relates to a sealant film, a package and a medical instrument using the laminate film.

### BACKGROUND ART

Heretofore, polyolefin film is used as various food or medical packaging materials by making use of its characteristic features such as inexpensiveness, resistance against heat or chemicals and easy heat sealability.

In food packaging materials, for example, an aluminum foil, a vinylidene chloride resin or a saponified product of ethylene-vinyl acetate copolymer resin is used as a gas barrier layer, the gas barrier layer is combined with a polyamide resin layer or a polyester resin layer, and a polyolefin-base resin such as polypropylene and polyethylene is used as a sealant layer. In medical packaging materials, a polyolefin-base resin is also used as a sealant layer.

With respect to the easily openable sealant film, a method of using a resin composition obtained by mixing polypropylene and polyethylene for the sealant layer, and a method of using a resin obtained by graft-polymerizing styrene to a polyolefin-base resin are known.

In the sealant film for packages which are produced through checkers sealing, such as medical instrument package, a resin composition obtained by mixing a linear low-density polyethylene with a low melting point polypropylene has been heretofore used for the sealant layer and thereon, a homopolypropylene as a substrate layer is laminated.

This sealant film is characterized in that when the package is opened, whitening occurs in the heat sealed portion and therefore, it is visible that the package was infallibly heat-sealed.

However, the sealant film has a problem in that since the heat seal strength (peal strength in the heat sealed portion) is as high as 11.8 N/15 mm-width or more, the package is difficult to open; since the heat seal temperature width is narrow, the checkers sealing machine fails in controlling the temperature; or when the heat seal pressure fluctuates, homopolypropylenes are heat-sealed with each other and the film is unopenable. The term “the heat seal temperature width is narrow” as used herein means that changes in the heat seal conditions such as heat seal temperature and heat seal pressure cause great change in the heat seal strength.

Examples of the easily openable sealant film include a package having a heat sealable resin layer which comprises a resin composition layer having a thickness of 5 to 25 μm and containing a crystalline polypropylene-base resin having a melt index of 2 to 100 g/10 min and a polyethylene having a melt index of 2 to 50 g/10 min in a weight ratio of 50:50 to 95:5 (see, Japanese Unexamined Patent Application, First Publication No. 58-1672), an easily openable drop lid comprising three layers of an uppermost layer formed of a heat-resistant resin layer, an intermediate layer formed of a polypropylene and a cohesive failure sealant layer provided under the intermediate layer and formed of a polypropylene-polyethylene blend polymer (see, Japanese Utility Model Application, Second Publication No. 61-37647, a container comprising a film layer formed of a blend material of a base resin comprising a crystalline polypropylene or a crystalline propylene-ethylene copolymer with from 8 to 30 wt % of an ethylene-vinyl acetate copolymer having a vinyl acetate content of 8 to 5 wt %, where the heat seal strength after the thermal sterilization is from 100 to 1,700 g/cm (see, Japanese Unexamined Patent Application, First Publication No. 57-28777), and a composite film comprising a propylene-base resin layer and a resin composition layer containing a propylene-ethylene random copolymer and/or a propylene-ethylene-butene-1 terpolymer, and a high-pressure polyethylene and/or a linear low-density polyethylene (see, Japanese Unexamined Patent Application, First Publication No. 63-179741).

These sealant films have a problem in that the low-temperature heat sealability is inferior, the open strength is too strong or the heat seal portion shows poor whitening or in an extreme case, shows utterly no whitening at the time of opening.

Examples of the case using a combination of a polyethylene and a polybutene-1 as in the present invention include a method of blending the polyethylene and the polybutene-1 for preventing distortion and cracking of a polyethylene-made bottle, pipe, electric wire coating or the like due to contact with a viscous liquid (see, Japanese Examined Patent Application, Second Publication No. 39-22351. However, this publication neither discloses nor suggests the object of the present invention or the method for solving the problems described in the present invention.

### DISCLOSURE OF INVENTION

The present invention has been made under these circumstances and the main object of the present invention is to provide a laminate film suitable as a food or medical packaging material which is excellent in easy openability, whitening property, water vapor barrier property and low-temperature heat sealability, has a wide heat seal temperature width and can be adapted also for checkers sealing. The object of the present invention includes providing a sealant film and a package using the laminate film.

As a result of extensive investigations, the present inventors have found that the above-described object can be attained by laminating a specific resin composition layer and a high-density polyethylene layer. The present invention has been accomplished based on this finding.

More specifically, the present invention relates to a laminate film, a sealant film and a package described in the following (1) to (11).

(1) A laminate film comprising at least two layers of a resin composition layer [layer (A)] containing (a) from 30 to 60 mass % of a linear low-density polyethylene produced using a metallocene catalyst or a single site-type catalyst, (b) from 10 to 30 mass % of a low-density polyethylene and (c) from 10 to 40 mass % of at least one resin selected from the group consisting of an ethylene-propylene copolymer, an ethylene-butene copolymer, a propylene-butene copolymer, an ethylene-propylene-butene terpolymer and polybutene-1 [provided that (a)+(b)+(c)=100 mass %], and a high-density polyethylene layer [layer (B)] containing (d) a high-density polyethylene having a density of 0.950 to 0.970 g/cm³.

(2) The laminate film as described in (1), wherein the linear low-density polyethylene (a) has a density of 0.890 to 0.935 g/cm³ and a melt flow rate of 2 to 20 g/10 min.

(3) The laminate film as described in (1) or (2), wherein the high-density polyethylene (c) has a melt flow rate of 1 to 15 g/10 min.

(4) A laminate film as described in (1), wherein a layer [layer (C)] containing (e) a linear low-density polyethylene produced using a metallocene catalyst or a single site-type catalyst is provided on the layer (B) in the opposite side to the layer (A).

(5) A method for producing the laminate film described in any one of (1) to (4), comprising forming a film at an extrusion temperature of 160 to 250° C. and cooling the film at a cooling temperature of 40 to 80° C.

(6) A sealant film using the layer (A) of the laminate film described in any one of (1) to (4) as a sealant layer.

(7) A packaging material comprising a gas barrier layer and/or a protective layer and the sealant film described in (6).

(8) A package using the layer (A) of the laminate film described in any one of (1) to (4) as a sealant layer.

(9) The package as described in (8), wherein the layer (A) and the layer (A) of the laminate films described in any one of (1) to (4) are melt-bonded to each other.

(10) A medical instrument packaged by the package described in (8) or (9).

(11) The medical instrument as described in (10), which is a blood sugar measuring electrode.

### BEST MODE FOR CARRYING OUT THE INVENTION

The present invention is described in detail below.

The linear low-density polyethylene (a) (hereinafter sometimes referred to as “component (a)”) used for the resin composition layer [layer (A)] in the present invention and the linear low-density polyethylene (e) (hereinafter sometimes referred to as “component (e)”) used for the layer (c) are a linear low-density polyethylene (hereinafter sometimes referred to as “mLLDPE”) produced using a metallocene catalyst or a single site-type catalyst.

The representative definition of the metallocene catalyst is described, for example, in Tetsuya Harada, “Metallocene Shokubai ni yoru Jisedai Polymer Kogyoka Gijutsu (Next Generation Polymer Industrialization Technology by Metallocene Catalyst)”, page 13, JISK (1994) or in “Olefin-kei, Styrene-kei Jushi no Kokinoka (Olefin-Base and Styrene-Base Resin Imparted with High Performance)”, page 13, Gijutsu Joho Kyokai (2000). However, the metallocene catalyst here includes, as generally defined, catalysts comprising a complex of a metal atom such as zirconium, titanium and hafnium with a compound having at least one cyclopentadienyl ring, namely, a cyclopentadienyl complex salt. Examples of the cocatalyst include boron compounds such as triphenyl(hydroxyphenyl) borate and tris-(pentafluorophenyl) (4-hydroxyphenyl) borate, and almoxanes such as methyl almoxane, however, the present invention is not limited thereto. A certain kind of metallocene catalysts are representative single site-type catalysts and hereinafter, the metallocene catalyst as used in the present invention means a metallocene catalyst and/or a single site-type catalyst.

The mLLDPE is a copolymer mainly comprising an ethylene and one or more olefin selected from α-olefins having from 3 to 20 carbon atoms. The α-olefin having from 3 to 20 carbon atoms is preferably an α-olefin having from 3 to 12 carbon atoms and specific examples thereof include propylene, 1-butene, 1-pentene, 4-methyl-1-pentene, 1-hexene, 1-octene, 1-decene and 1-dodecene. The content of the α-olefin having from 3 to 20 carbon atoms occupying in the copolymer is usually 30 mol % or less, preferably from 2 to 20 mol %.

The density (measured according to JIS K 7112D method) of the mLLDPE for use in the present invention is preferably from 0.890 to 0.935 g/cm³, more preferably from 0.892 to 0.933 g/cm³, still more preferably from 0.894 to 0.931 g/cm³. If the density is less than 0.890 g/cm³, the heat seal strength may be too strong, whereas if it exceeds 0.935 g/cm³, a feathering phenomenon is liable to occur at the opening.

The melt flow rate (hereinafter referred to as “MFR”) is preferably from 2 to 25 g/10 min, more preferably from 6 to 22 g/10 min, still more preferably from 7 to 20 g/10 min. If the MFR is less than 2 g/10 min, too strong heat seal strength and poor whitening property are liable to result, whereas if it exceeds 20 g/10 min, the compatibility with polybutene is poor and a feathering phenomenon may occur or poor whitening property may result. The MFR is a value measured at a temperature of 190° C. under a load of 21.18N according to Test Condition 4 of JIS K 7210.

The low-density polyethylene (b) for use in the present invention (hereinafter sometimes referred to as “LDPE” or simply as “component (b)”) is not particularly limited but preferably has a density of 0.910 to 0.925 g/cm³ as measured according to JIS K7112 Method D and an MFR of 20 g/10 min or less as measured according to Test Condition 4 of JIS K7210. The density is preferably from 0.912 to 0.924 g/cm³, more preferably from 0.915 to 0.923 g/cm³. The MFR is preferably 15 g/10 min or less, more preferably from 0.1 to 10 g/10 min.

In the present invention, the layer (A) contains at least one resin [hereinafter sometimes referred to as “component (c)”] selected from the group consisting of an ethylene-propylene copolymer, an ethylene-butene copolymer, a propylene-butene copolymer, an ethylene-propylene-butene terpolymer and polybutene-1. Among these resins, preferred are butene-base copolymers such as ethylene-butene copolymer, propylene-butene copolymer, ethylene-propylene-butene terpolymer and polybutene-1, more preferred is polybutene-1.

The ethylene-propylene copolymer for use in the present invention preferably has an ethylene content of 65 to 85 mass % and an MFR of 0.2 to 20 g/10 min as measured at a temperature of 230° C. under a load of 21.18N according to JIS K7210. If the ethylene content is 60 mass % or less, the low-temperature sealability and the whitening property are inferior, whereas if it exceeds 85 mass %, the easy openability deteriorates. The ethylene content is preferably from 65 to 82 mass %, more preferably from 70 to 80 mass %. If the MFR is less than 0.2 g/10 min, the easy openability deteriorates, whereas if it exceeds 20 g/10 min, the whitening property and the formability are inferior. The MFR is preferably from 0.2 to 18 g/10 min, more preferably from 0.5 to 15 g/10 min.

The ethylene-butene copolymer for use in the present invention preferably has an ethylene content of 70 to 98 mass % and an MFR of 0.1 to 20 g/10 min as measured at a temperature of 230° C. under a load of 21.18N according to JIS K7210. If the ethylene content is less than 70 mass %, the heat resistance, the peel appearance and the whitening property are disadvantageously inferior, whereas if it exceeds 98 mass %, the easy openability and the whitening property deteriorate. The ethylene content is preferably from 72 to 96 mass %, more preferably from 75 to 95 mass %. If the MFR is less than 0.1 g/10 min, the easy openability deteriorates, whereas if it exceeds 10 g/10 min, the peel appearance and the whitening property are disadvantageously inferior. The MFR is preferably from 0.2 to 9 g/10 min, more preferably from 0.5 to 8 g/10 min.

The propylene-butene copolymer for use in the present invention preferably has a propylene content of 75 to 98 mass % and an MFR of 0.1 to 20 g/10 min as measured at a temperature of 230° C. under a load of 21.18N according to JIS K7210. If the propylene content is less than 75 mass %, the easy openability and the water vapor barrier property deteriorate, whereas if it exceeds 98 mass %, the easy openability, the whitening property and the low-temperature heat sealability are disadvantageously inferior. If the MFR is less than 0.1 g/10 min, the easy openability deteriorates, whereas if it exceeds 20 g/10 min, the peel appearance and the whitening property are disadvantageously inferior. The MFR is preferably from 0.5 to 18 g/10 min, more preferably from 1 to 15 g/10 min.

The ethylene-propylene-butene terpolymer (hereinafter sometimes referred to as “PEB”) for use in the present invention is a copolymer mainly comprising propylene, ethylene and butane-1, and this resin is generally obtained by a multistage copolymerization process.

The PEB for use in the present invention preferably has a crystallization peak temperature (Tcp) of 75 to 90° C. and a crystallization energy (ΔH) of 55 to 80 J/g, as measured by a differential scanning calorimeter (DSC).

The melting temperature (Tmp) is not particularly limited but is preferably from 125 to 145° C.

The Tcp and ΔH as used herein are determined by setting a sample exactly weighed to 3 to 5 mg on a DSC, elevating the sample temperature up to 230° C., holding the sample for 5 minutes and then, lowering the temperature to 30° C. at a rate of 20° C./min.

The composition ratio of the PEB is preferably such that the propylene content is from 96 to 72 mass %, the ethylene content is from 1 to 10 mass % and the butane-1 content is from 3 to 18 mass %. The propylene content is preferably from 95.3 to 75 mass %, more preferably from 94.5 to 77 mass %. The ethylene content is preferably from 1.2 to 8 mass %, more preferably from 1.5 to 7 mass %. The butane-1 content is preferably from 3.5 to 17 mass %, more preferably from 4.0 to 16 mass %.

The polybutene-1 (hereinafter referred to as a “PB”) for use in the present invention is obtained by the polymerization starting from a butene-1 monomer and is the same polyolefin as polyethylene and polypropylene. In general, the PB has a high molecular weight and a high isotacticity. For the polymerization, a polymerization method using a Ziegler-Natta catalyst and a butene-1 monomer as a solvent, or a solution polymerization method using a hydrocarbon-base solvent such as hexane and heptane, is used.

The PB is known as a polymer having rubber elasticity because due to its second order transition temperature lower than that of polypropylene, the elastic modulus thereof at room temperature or low temperatures is higher than that of polypropylene [see, D. G. Natta et. al., J. Polymer Sci., Vol. 25, p. 119 (1957)].

As compared with polyethylene or polypropylene, the PB is excellent in the mechanical properties such as tensile strength, impact strength and tear strength, and therefore, is used as a pipe material, a tube or a modifier.

In the field of film, the PB is used by blending it with another resin so as to impart low-temperature heat sealability or easy peelability.

These properties and uses of PB are described in detail, for example, in “Saishin Laminate Kako Binran (Handbook of Newest Laminate Processing)”, pp. 861-866 (1989), Kako Gijutsu Kenkyu Kai, and I. D. Rubin, Poly(1-Butene)—its Preparation and Properties, Gordon and Breach Science Publishers, Inc., New York (1968).

The PB for use in the present invention preferably has an MFR of 0.5 to 15 g/10 min, more preferably from 0.8 to 12 g/10 min, most preferably from 1 to 10 g/10 min, as measured according to ASTM D1238E. If the MFR is less than 0.5 g/10 min, the heat seal strength is disadvantageously too high, whereas if it exceeds 15 g/10 min, a feathering phenomenon may occur in the portion where the sealed portion is peeled, or bad whitening property may result at the peeling.

The melting point of the PB is preferably 100 to 140° C. as measured by a method using a differential scanning calorimeter (DSC). If the melting point is less than 100° C., a feathering phenomenon may occur in the portion where the sealed portion is peeled, or poor whitening property may result at the peeling, whereas if it exceeds 140° C., the heat seal strength and the whitening property are liable to deteriorate.

The PB is available on the market, for example, under the trade name of “BEAULON” from Mitsui Chemicals and Shell Chemical, and these PBs can be suitably used.

The blend ratio among the component (a), the component (b) and the component (c) constituting the layer (A) is such that the component (a) is from 30 to 60 mass %, the component (b) is from 10 to 30 mass % and the component (c) is from 10 to 40 mass % (provided that (a)+(b)+(c)=100 mass %), preferably such that the component (a) is from 35 to 55 mass %, the component (b) is from 13 to 28 mass % and the component (c) is from 15 to 38 mass %, more preferably such that the component (a) is from 40 to 52 mass %, the component (b) is from 15 to 25 mass % and the component (c) is from 18 to 35 mass %.

If the component (a) is less than 30 mass %, reduction may be caused in the water vapor barrier property, the heat seal strength and the whitening property and also, a feathering phenomenon is disadvantageously liable to occur at the peeling, whereas if it exceeds 60 mass %, the whitening property deteriorates or at the heat sealing, sealing occurs also in the periphery of the seal portion to cause problems, for example, the appearance of the package is impaired or the content is difficult to take out.

If the component (b) is less than 10 mass %, the whitening property and the easy openability are inferior or a feathering phenomenon is disadvantageously liable to occur at the peeling, whereas if it exceeds 30 mass %, the formability and the heat seal strength may be deteriorated.

If the component (c) is less than 10 mass %, the easy openability deteriorates, whereas if it exceeds 40 mass %, a feathering phenomenon is disadvantageously liable to occur at the peeling or the water vapor barrier property is poor.

The high-density polyethylene (d) (hereinafter sometimes referred to as a “HDPE”) for use in the high-density polyethylene layer [layer (B)] has a density of 0.950 to 0.970 g/cm³, preferably from 0.952 to 0.965 g/cm³, more preferably from 0.953 to 0.962 g/cm³. If the density is less than 0.950 g/cm³, the water vapor barrier property or the whitening property at the peeling deteriorates, whereas if it exceeds 0.970 g/cm³, the low-temperature heat sealability or the whitening property is disadvantageously impaired.

The MFR of the HDPE is preferably from 1 to 15 g/10 min, more preferably from 2 to 13 g/10 min, still more preferably from 3 to 12 g/10 min, as measured at a temperature of 190° C. under a load of 21.18N according to JIS K7210. If the MFR is less than 1 g/10 min, the low-temperature heat sealability and the whitening property are liable to deteriorate, whereas if it exceeds 15 g/10 min, the film readily tears in the machine direction or a feathering phenomenon may occur at the peeling.

The method for producing the laminate film of the present invention is not particularly limited and a known method may be used. Examples thereof include a method of adding, if desired, additives to the resin component for each layer of the laminate film, mixing respective components by a mixer such as mixing roll, Banbury mixer, Henschel mixer, tumbler or ribbon blender, then kneading and pelletizing the mixture using an extruder or directly dry-blending the mixture, and forming a film by extrusion lamination, co-extrusion inflation, co-extrusion casting or the like.

In the case of obtaining the laminate film comprising at least two layers of the layer (A) and the layer (B) or further having the layer (C) in the layer (B) side of the present invention by a co-extrusion method, the film is preferably formed at an extrusion temperature of 160 to 250° C. and cooled at a cooling temperature of 40 to 80° C., so that a laminate film having good water vapor barrier property and excellent whitening property can be obtained.

For satisfying the object of the present invention, the laminate film of the present invention has a thickness of 20 to 70 μm, preferably from 20 to 60 μm, more preferably from 25 to 50 μm. Examples of the layer structure include layer (A)/layer (B), layer (A)/layer (B)/layer (A), and layer (A)/layer (B)/layer (C). In particular, the laminate film preferably comprises a three-kind three-layer structure of layer (A)/layer (B)/layer (C) because the film is reduced in the curling.

The thickness of the layer (A) is suitably from 3 to 15 μm by taking account of the invasion of water vapor from the end face of the package produced.

The thickness ratio between the layer (A) and the layer (B) is suitably layer (A):layer (B)=0.05 to 0.3:0.95 to 0.7, preferably 0.07 to 0.25:0.93 to 0.75, more preferably 0.1 to 0.2:0.9 to 0.8.

If the thickness ratio of the layer (A) is 0.05 or less, the layer (B) and the layer (B) are partially heat-sealed when heat-sealing is applied and this may cause a problem such that the heat seal strength elevates or on opening, the laminate film is cut at the heat seal end part, as a result, the content cannot be easily taken out. On the other hand, if the thickness ratio of the layer (A) exceeds 0.3, feathering may take place or the package may deteriorate in the water vapor barrier property. The thickness of the layer (C) can be appropriately selected so as to prevent curling. Specifically, for example, the thickness of the layer (C) is preferably on the same level as the layer (A).

In the sealant film of the present invention, the layer (A) of the laminate film comprising at least two layers of layer (A) and layer (B) or the laminate film having the layer (C) in the layer (B) side of the above-described laminate film is used as the sealant layer.

On peeling the package where the layer (A) comprising a resin composition is used for the sealant layer, cohesive peeling due to cohesive failure is generated inside the resin composition layer comprising the components (a), (b) and (c) each in a specific amount, whereby excellent easy openability and excellent whitening property are revealed.

Although the reason is not clearly known, the excellent whitening property as one advantage of the present invention is presumed attributable to the fact that the component (c) constituting the resin composition layer has a higher molecular weight and a higher cohesive energy than other components and therefore, when the package is peeled, fine voids are generated at the interface between the component (c) and other components.

In the sealant film of the present invention, a layer comprising HDPE must be laminated on the sealant layer.

This is because if a layer comprising HDPE is not laminated on the sealant layer, the water vapor barrier property or heat seal strength stability may deteriorate or in an extreme case, the periphery of the heat seal portion melts to cause bonding and the content of the package cannot be easily taken out.

Accordingly, the package excellent in the easy openability, whitening property, water vapor barrier property and low-temperature heat sealability and having a wide heat seal temperature width, which is one of the objects of the present invention, can be obtained by using a laminate film where the layer comprising HDPE is laminated on the sealant layer.

The packaging material of the present invention comprises the sealant film of the present invention, a gas barrier layer and/or a protective layer. The gas barrier layer and the protective layer can be laminated on the sealant film through or not through an adhesive. Examples of the gas barrier layer include layers comprising an aluminum foil, a metallized film, a silicon oxide-deposited film, a vinylidene chloride resin or a saponified product of ethylene-vinyl acetate copolymer. Specific examples of the protective layer include a polyester resin layer, a polyamide resin layer and a polycarbonate resin layer. Other polyolefin-base resin layers may also be further laminated within the range of not impairing the object of the present invention. The packaging material of the present invention can be obtained by a known method such as extrusion lamination or dry lamination.

The package of the present invention is characterized in that the layer (A) of the laminate film of the present invention is used as the sealant layer. The sealing object bonded to the layer (A) may be a film or a container. The material of the sealing object is not particularly limited as long as it can be bonded to the layer (A), however, polyolefin is preferred. Specific examples of the polyolefin include homopolymers of olefin, such as polyethylene, polypropylene, polybutene and poly-4-methylpentene-1; copolymers of α-olefin (e.g., 1-butene, 1-hexene, 1-octene) and ethylene or propylene; copolymers of olefin and α-olefin, such as ethylene-propylene copolymer; copolymers (including a graft copolymer) of olefin (e.g., ethylene, propylene) and unsaturated dicarboxylic acid anhydride (e.g., maleic anhydride, itaconic anhydride, citraconic anhydride, endobicyclo-[2,2,1]-hept-5-ene-2,3-dicarboxylic acid anhydride); copolymers of ethylene and unsaturated carboxylic acid or an unsaturated carboxylic acid ester, such as ethylene-(meth)acrylic acid copolymer and ethylene-(meth)acrylic acid ester copolymer; and ethylene-base terpolymers such as ethylene-(meth)acrylic acid ester-maleic anhydride copolymer.

A preferred embodiment of the package of the present invention is a package where the layer (A) and the layer (A) are fused using the laminate films of the present invention. This package is very excellent in the easy openability, whitening property, water vapor barrier property and low-temperature heat sealability and has a wide heat seal temperature width.

The constituent materials of each layer constituting the present invention may be appropriately blended with other additives commonly used for the thermoplastic resin (for example, antioxidant, weather resistance stabilizer, antistatic agent, lubricant, blocking inhibitor, anti-whitening agent, dye, pigment, oil, wax and filler) or other thermoplastic resins within the range of not impairing the object of the present invention.

Examples of the additives include, as an antioxidant, 2,5-di-tert-butylhydroquinone, 2,6-di-tert-butyl-p-cresol, 4,4′-thiobis(6-tert-butylphenol), 2,2-methylene-bis(4-methyl-6-tert-butylphenol), octadecyl-3-(3′,5′-di-tert-butyl-1′-hydroxyphenyl)propionate and 4,4′-thiobis(6-butylphenol); as an ultraviolet absorbent, ethyl-2-cyano-3,3-diphenylacrylate, 2-(2′-hydroxy-5-methylphenyl)benzotriazole and 2-hydroxy-4-octoxybenzophenone; as a plasticizer, dimethyl phthalate, diethyl phthalate, wax, liquid paraffin and phosphoric acid ester; as an antistatic agent, pentaerythritol monostearate, sorbitan monopalmitate, sulfated oleic acid, polyethylene oxide and carbon wax; as a lubricant, ethylenebisstearamide, butyl stearate and fatty amide (e.g., stearic acid amide, oleic amide, erucic amide); as a coloring agent, carbon black, phthalocyanine, quinacridone, indoline, azo-type pigment, titanium oxide and red iron oxide; as a filler, glass fiber, asbestos, mica, wollastonite, calcium silicate, aluminum silicate and calcium carbonate; and as an antiblocking agent, silicate (e.g., calcium silicate, aluminum silicate), silicone, zeolite, talc and polymethyl methacrylate (PMMA) particle. As for the addition method and amount added, in a specific example, from 6,000 to 12,000 ppm of silicate, from 2,000 to 6,000 ppm of PMMA particle and from 300 to 1,000 ppm of fatty amide are added to the layer (A) and from 4,000 to 8,000 ppm of silicate is added to the layer (C), whereby the main purpose of the present invention can be satisfied and a laminate film further having excellent blocking resistance can be obtained. In the case where the additives are in the particle form, the average particle size is preferably from 0.05 to 20 μm, more preferably from 0.1 to 10 μm.

The package using the laminate film of the present invention is characterized in that when the package is opened, the heat sealed portion is whitened and therefore, it is easily visible that the package was infallibly heat-sealed. Accordingly, the package can be suitably used as a food or medical packaging material. In particular, the package can be preferably used as a packaging material for medical instrument where easy openability and water vapor barrier property are necessary in addition to the whitening property and a strict management is required. Examples of the medical instrument include a blood sugar measuring electrode. One preferred embodiment of the present invention is a blood sugar measuring electrode packaged through checkers sealing.

### EXAMPLES

The present invention is described in greater detail below by referring to Examples, however, the present invention is not limited to these Examples.

[Resin Used]

PE 1:

A single site-type catalyst linear low-density polyethylene having an MFR of 15 g/10 min and a density of 0.912 g/cm³.

PE 2:

A single site-type catalyst linear low-density polyethylene having an MFR of 8 g/10 min and a density of 0.907 g/cm³.

PE 3:

A high-pressure low-density polyethylene using a peroxide, having an MFR of 5.0 g/10 min and a density of 0.918 g/cm³.

PE 4:

A Ziegler-Natta type linear low-density polyethylene having an MFR of 2 g/10 min and a density of 0.929 g/cm³.

PE: 5

A high-pressure low-density polyethylene using a peroxide having an MFR of 3.5 g/10 min and a density of 0.924 g/cm³.

PE 6:

A single site-type catalyst linear low-density polyethylene having an MFR of 3.5 g/10 min and a density of 0.921 g/cm³.

PE 7:

A single site-type catalyst linear low-density polyethylene having an MFR of 3.5 g/10 min and a density of 0.915 g/cm³.

PE 8:

A Ziegler-Natta type linear low-density polyethylene having an MFR of 2.8 g/10 min and a density of 0.932 g/cm³.

HDPE 1:

A high-density polyethylene having an MFR of 7.6 g/10 min and a density of 0.958 g/cm³.

HDPE 2:

A high-density polyethylene having an MFR of 10.8 g/10 min and a density of 0.962 g/cm³.

HDPE 3:

A high-density polyethylene having an MFR of 3.6 g/10 min and a density of 0.964 g/cm³.

HDPE 4:

A high-density polyethylene having an MFR of 8.6 g/10 min and a density of 0.945 g/cm³.

HDPE 5:

A high-density polyethylene having an MFR of 8.5 g/10 min and a density of 0.960 g/cm³.

PB 1:

“BEAULON BL4000 (trade name)” produced by Mitsui Chemicals Inc., which is a polybutene-1 having an MFR of 1.8 g/10 min, a density of 0.915 g/cm³ and a melting point of 125° C.

PB 2:

“BEAULON BL7000 (trade name)” produced by Mitsui Chemicals Inc., which is a polybutene-1 having an MFR of 20 g/10 min, a density of 0.917 g/cm³ and a melting point of 123° C.

PB 3:

“BEAULON M2181 (trade name)” produced by Mitsui Chemicals Inc., which is a polybutene-1 having an MFR of 1.0 g/10 min, a density of 0.900 g/cm³ and a melting point of 75° C.

PEP:

“TAFMER P (trade name)” produced by Mitsui Chemicals Inc., which is an ethylene-propylene copolymer having an MFR of 5.4 g/10 min and a density of 0.87 g/cm³.

PEB:

“TAFMER A (trade name)” produced by Mitsui Chemicals Inc., which is an ethylene-propylene copolymer having an MFR of 3.6 g/10 min and a density of 0.88 g/cm³.

PPB:

“Clyrell 722RCXP (trade name)” produced by Sun Alomer Ltd., which is a propylene-butene copolymer having an MFR of 10 g/10 min and a density of 0.90 g/cm³.

PEPB:

“Adsyl 5C37F (trade name)” produced by Sun Alomer Ltd., which is an ethylene-propylene-butene terpolymer having an MFR of 5.5 g/10 min and a density of 0.90 g/cm³.

PP 1:

A propylene-ethylene random copolymer having an ethylene content of 6.5% by weight and an MFR of 20 g/10 min at a temperature of 230° C. under a load of 2.16 kg.

PP 2:

A homopolypropylene having an MFR of 8.5 g/10 min.

<Preparation of Specimen>

[Kneading Treatment]

The components (a), (b) and (c) as constituent materials of the layer (A) were mixed in a tumbler and then the mixture was pelletized at a temperature of 190 to 210° C. using a twin extruder (Model KTX37, manufactured by Kobe Steel, Ltd.). At this time, 4,000 ppm of PMMA particle was added as an antiblocking agent.

[Cast Molding]

Using each kneaded pellet material, a film was prepared at a die temperature of 210° C. and a cooling temperature of 70° C. by means of a casting film molding machine having an aperture of 65-mmφ and a die width of 1,300 mm manufactured by Toshiba Machine Co., Ltd. while appropriately varying the total thickness. The layer structure of the film was a three-kind three-layer structure of layer (A)/layer (B)/layer (C). The thickness was layer (A)/layer (B)/layer (C)=6 μm/28 μm/6 μm.

On each film, a 12 μm-thick polyester film and a 15 μm-thick aluminum foil were laminated by a dry lamination process. The layer (A) and the layer (A) of the thus-obtained laminate films were heat-sealed and the obtained package was evaluated as follows.

[Heat Seal]

Using a heat sealer manufactured by Tester Sangyo K.K., a heat-seal treatment was performed by a double cut heat-seal bar having an asperity inclination angle of 55° under a pressure of 0.2 MPa for 1 second.

<Measuring Method of Physical Properties>

[Heat Seal Strength]

A film after heat-sealing was cut into a width of 15 mm and determined on the 180° peel strength at a pulling rate of 300 mm/min using a tension tester (Model RTA-100, manufactured by Olientech). In the test, an average of 10 specimens was determined. The sealing temperature was appropriately varied.

[Whitening Property]

After the determination of heat-seal strength, each specimen was observed with an eye on the whitened state in the peeled heat-seal portion. Evaluation was performed according to the following criteria.

◯: Thoroughly whitened and the whitening was easily confirmed.

Δ: Slightly whitened and the whitening was not easily confirmed.

X: Not whitened at all.

[Appearance]

After the measurement of the heat-seal strength, the peeled portion was judged with an eye whether or not feathering occurred, according to the following criteria.

◯: Feathering was not observed at all.

Δ: Feathering was partially observed.

X: Conspicuous feathering and poor appearance.

[Water Vapor Transmission Rate]

In accordance with ASTM E96, the water vapor transmission rate at a temperature of 37.8° C. and a relative humidity of 90% was measured using a water vapor permeation tester PERMATRAN-W manufactured by Modern Control Ltd.

[Evaluation of Antiblocking Property]

The films were superposed and stored at a temperature of 40° C. for 2 days while applying thereon a load of 98 N/100 cm². Thereafter, the films were cooled to 23° C. and determined on the peeling strength at a peeling rate of 500 mm/min using a tension tester. As the peeling strength is larger, the blocking strength between films is higher.

### Examples 1 to 13, Comparative Examples 1 to 4

Using the laminate films shown in Table 1, the heat sealability, the water vapor transmission rate and the antiblocking property were measured. The results obtained are shown in Table 1. In the layer (A), 8,000 ppm of aluminum silicate having an average particle size of 0.8 μm, 4,000 ppm of PMMA particle having an average particle size of 2 μm and 600 ppm of erucic acid amide were blended. In the layer (C), 6,000 ppm of aluminum silicate having an average particle size of 0.8 μm was blended.

### Example 14

The measurements were performed in the same manner as in Example 5 except that 4,500 ppm of aluminum silicate, 3,000 ppm of PMMA particle and 350 ppm of erucic acid amide were blended in the layer (A) and 4,500 ppm of aluminum silicate having an average particle size of 0.8 μm was blended in the layer (C). The results obtained are shown in Table 1.

### Example 15

The measurements were performed in the same manner as in Example 5 except that 10,500 ppm of aluminum silicate, 5,500 ppm of PMMA particle and 950 ppm of erucic acid amide were blended in the layer (A) and 7,500 ppm of aluminum silicate having an average particle size of 0.8 μm was blended in the layer (C). The results obtained are shown in Table 1.

### Examples 16 to 19 and Comparative Examples 5 and 6

The layer (A) and the layer (A) of laminate films were heat-sealed by changing the total thickness and the thickness structure of respective layers in each of the laminate films of Example 5 and Comparative Examples 2 and 4. Thereafter, the measurements were performed in the same manner as in Example 5. The results obtained are shown in Table 2.

TABLE 1

| Exam- | | | | | | | | | | | | | | | |
|---|---|---|---|---|---|---|---|---|---|---|---|---|---|---|---|
| ple or | Laminate Film | | | | | | | | | | | | | Water | Anti- |
| Com- | Layer (A) | | | | | | | | | | | | | Vapor | block- |
| para- | Component | | Component | | Component | | | | Heat Seal Property | | | | | Trans- | ing |
| tive | (a) | | (b) | | (c) | | | | | | | Whiten- | | mission | Prop- |
| Exam- | | Amount | | Amount | | Amount | | | Seal Strength | | | ing | | Rate | erty |
| ple | | Blended | | Blended | | Blended | Layer | Layer | (N/15 mm width) | | | Prop- | Appear- | (g/m² · | (g/100 |
| No. | Kind | (mass %) | Kind | (mass %) | Kind | (mass %) | (B) | (C) | 120° C. | 140° C. | 160° C. | erty | ance | day) | cm²) |
| Exam- | PE 1 | 50 | PE 3 | 15 | PB 1 | 35 | HDPE 1 | PE 6 | 4.31 | 7.06 | 9.21 | ◯ | ◯ | 5.3 | 60 |
| ple 1 | | | | | | | | | | | | | | | |
| Exam- | PE 1 | 55 | PE 3 | 20 | PB 1 | 25 | HDPE 1 | PE 6 | 5.68 | 7.35 | 9.02 | ◯ | ◯ | 5.1 | 30 |
| ple 2 | | | | | | | | | | | | | | | |
| Exam- | PE 1 | 60 | PE 3 | 25 | PB 1 | 15 | HDPE 1 | PE 6 | 4.21 | 6.37 | 10.3 | ◯ | ◯ | 5.2 | 25 |
| ple 3 | | | | | | | | | | | | | | | |
| Exam- | PE 1 | 40 | PE 3 | 25 | PB 1 | 35 | HDPE 1 | PE 6 | 4.41 | 4.99 | 7.74 | ◯ | ◯ | 5.6 | 35 |
| ple 4 | | | | | | | | | | | | | | | |
| Exam- | PE 1 | 50 | PE 5 | 20 | PB 1 | 30 | HDPE 1 | PE 6 | 5.19 | 5.68 | 8.04 | ◯ | ◯ | 5.5 | 30 |
| ple 5 | | | | | | | | | | | | | | | |
| Exam- | PE 1 | 50 | PE 3 | 20 | PB 2 | 30 | HDPE 2 | PE 6 | 4.70 | 5.09 | 7.35 | ◯ | ◯ | 5.8 | 30 |
| ple 6 | | | | | | | | | | | | | | | |
| Exam- | PE 1 | 50 | PE 3 | 20 | PB 3 | 30 | HDPE 3 | PE 7 | 5.09 | 6.66 | 10.8 | Δ | ◯ | 5.4 | 30 |
| ple 7 | | | | | | | | | | | | | | | |
| Exam- | PE 2 | 50 | PE 5 | 20 | PB 2 | 30 | HDPE 5 | PE 7 | 4.70 | 5.19 | 6.66 | ◯ | ◯ | 6.7 | 30 |
| ple 8 | | | | | | | | | | | | | | | |
| Exam- | PE 1 | 50 | PE 3 | 20 | PB 1 | 30 | HDPE 1 | PE 6 | 4.70 | 5.68 | 8.04 | Δ | ◯ | 6.9 | 30 |
| ple 9 | | | | | | | | | | | | | | | |
| Exam- | PE 1 | 50 | PE 3 | 20 | PEP | 30 | HDPE 1 | PE 6 | 3.72 | 4.41 | 6.37 | Δ | Δ | 6.2 | 70 |
| ple 10 | | | | | | | | | | | | | | | |
| Exam- | PE 1 | 50 | PE 3 | 20 | PEB | 30 | HDPE 1 | PE 6 | 4.02 | 5.19 | 7.06 | ◯ | Δ | 5.8 | 60 |
| ple 11 | | | | | | | | | | | | | | | |
| Exam- | PE 1 | 50 | PE 3 | 20 | PPB | 30 | HDPE 1 | PE 6 | 3.52 | 4.21 | 9.41 | Δ | ◯ | 5.9 | 25 |
| ple 12 | | | | | | | | | | | | | | | |
| Exam- | PE 1 | 50 | PE 3 | 20 | PEBP | 30 | HDPE 1 | PE 6 | 6.66 | 8.72 | 10.8 | Δ | ◯ | 6.2 | 50 |
| ple 13 | | | | | | | | | | | | | | | |
| Exam- | PE 1 | 50 | PE 3 | 20 | PB 1 | 30 | HDPE 1 | PE 6 | 5.10 | 5.49 | 7.74 | ◯ | ◯ | 5.3 | 45 |
| ple 14 | | | | | | | | | | | | | | | |
| Exam- | PE 1 | 50 | PE 3 | 20 | PB 1 | 30 | HDPE 1 | PE 6 | 5.19 | 5.59 | 7.84 | ◯ | ◯ | 5.4 | 25 |
| ple 15 | | | | | | | | | | | | | | | |
| Com- | PE 4 | 50 | PE 3 | 20 | PB 1 | 30 | HDPE 1 | PE 6 | 8.62 | 9.60 | 18.1 | X | X | 9.6 | 70 |
| para- | | | | | | | | | | | | | | | |
| tive | | | | | | | | | | | | | | | |
| Exam- | | | | | | | | | | | | | | | |
| ple 1 | | | | | | | | | | | | | | | |
| Com- | PP 1 | 50 | PE 3 | 20 | PE 4 | 20 | PP 2 | PP 2 | 2.74 | 3.04 | 3.43 | X | X | 12.2 | 85 |
| para- | | | | | | | | | | | | | | | |
| tive | | | | | | | | | | | | | | | |
| Exam- | | | | | | | | | | | | | | | |
| ple 2 | | | | | | | | | | | | | | | |
| Com- | PE 1 | 90 | | | PB 2 | 10 | HDPE 4 | PE 6 | 1.47 | 1.76 | 27.4 | X | X | 7.4 | 45 |
| para- | | | | | | | | | | | | | | | |
| tive | | | | | | | | | | | | | | | |
| Exam- | | | | | | | | | | | | | | | |
| ple 3 | | | | | | | | | | | | | | | |
| Com- | PE 3 | 20 | PE 4 | 40 | PB 3 | 40 | HDPE 4 | PE 8 | 15.2 | 22.1 | 31.7 | X | X | 13.4 | 195 |
| para- | | | | | | | | | | | | | | | |
| tive | | | | | | | | | | | | | | | |
| Exam- | | | | | | | | | | | | | | | |
| ple 4 | | | | | | | | | | | | | | | |

TABLE 2

| Example or | | Total | Layer Structure | | | Heat Seal Property | | | | | Water Vapor | Anti- |
|---|---|---|---|---|---|---|---|---|---|---|---|---|
| Comparative | | Thick- | Thickness (μm) | | | | | | Whiten- | | Trans- | blocking |
| Example | Kind of | ness | Layer | Layer | Layer | Seal Strength | | | ing | Appear- | mission Rate | Property |
| No. | Film | (μm) | (A) | (B) | (C) | 120° C. | 140° C. | 160° C. | Property | ance | (g/m² · day) | (g/100 cm²) |
| Example 16 | Example 5 | 30 | 5 | 20 | 5 | 5.29 | 5.59 | 8.23 | ◯ | ◯ | 5.4 | 40 |
| Example 17 | Example 5 | 40 | 10 | 20 | 10 | 5.19 | 5.00 | 7.64 | ◯ | ◯ | 5.1 | 55 |
| Example 18 | Example 5 | 50 | 15 | 20 | 15 | 4.90 | 5.68 | 8.43 | ◯ | ◯ | 5.5 | 75 |
| Example 19 | Example 5 | 40 | 18 | 4 | 18 | 5.39 | 5.98 | 8.04 | ◯ | Δ | 9.6 | 85 |
| Comparative | Comparative | 30 | 5 | 20 | 5 | 0.78 | 1.27 | 25.9 | X | X | 12.5 | 110 |
| Example 5 | Example 2 | | | | | | | | | | | |
| Comparative | Comparative | 40 | 10 | 20 | 10 | 14.2 | 21.1 | 28.0 | X | X | 13.8 | 265 |
| Example 6 | Example 4 | | | | | | | | | | | |

### INDUSTRIAL APPLICABILITY

The laminate film, the sealant film and the package of the present invention exhibit, when used as a sealant film, excellent easy openability, good whitening property, high water vapor barrier property, superior low-temperature heat sealability and wide heat seal temperature width as compared with conventional films, and is suitable as a food or medical packaging material.

In particular, the laminate film, the sealant film and the package of the present invention are suitable as a sealant film for a package which is produced through checkers sealing, such as a package for sugar blood measuring electrode.

1. A laminate film comprising at least two layers of a resin composition layer [layer (A)] containing (a) from 30 to 60 mass % of a linear low-density polyethylene produced using a metallocene catalyst or a single site-type catalyst, (b) from 10 to 30 mass % of a low-density polyethylene and (c) from 10 to 40 mass % of at least one resin selected from the group consisting of an ethylene-propylene copolymer, an ethylene-butene copolymer, a propylene-butene copolymer, an ethylene-propylene-butene terpolymer and polybutene-1 [provided that (a)+(b)+(c)=100 mass %], and a high-density polyethylene layer [layer (B)] containing (d) a high-density polyethylene having a density of 0.950 to 0.970 g/cm³.

2. The laminate film as claimed in claim 1, wherein the linear low-density polyethylene (a) has a density of 0.890 to 0.935 g/cm³ and a melt flow rate of 2 to 20 g/10 min.

3. The laminate film as claimed in claim 1, wherein the high-density polyethylene (c) has a melt flow rate of 1 to 15 g/10 min.

4. The laminate film as claimed in claim 1, wherein a layer [layer (C)] containing (e) a linear low-density polyethylene produced using a metallocene catalyst or a single site-type catalyst is provided on the layer (B) in the opposite side to the layer (A).

5. A method for producing the laminate film claimed in any one of claims 1 to 4, comprising forming a film at an extrusion temperature of 160 to 250° C. and cooling the film at a cooling temperature of 40 to 80° C.

6. A sealant film using the layer (A) of the laminate film claimed in any one of claims 1 to 4 as a sealant layer.

7. A packaging material comprising a gas barrier layer and/or a protective layer and the sealant film claimed in claim 6.

8. A package using the layer (A) of the laminate film claimed in claim 1 as a sealant layer.

9. The package as claimed in claim 8, wherein the layer (A) and the layer (A) of the laminate films claimed in claim 1 are melt-bonded to each other.

10. A medical instrument packaged by the package claimed in claim 8.

11. The medical instrument as claimed in claim 10, which is a blood sugar measuring electrode.
